(19) United States
(12) Patent Application Publication
(10) Pub. No.: US 2013/0273646 A1
(43) Pub. Date: Oct. 17, 2013

(54) INCUBATOR AND CARRIER SYSTEM

(71) Applicant: SANYO ELECTRIC CO., LTD., Osaka (JP)

(72) Inventors: Masahiko KOBAYASHI, Oizumi-machi (JP); Mikio HOUJOU, Osaka (JP); Akihito KOTERA, Kyoto-shi (JP)

(21) Appl. No.: 13/917,252

(22) Filed: Jun. 13, 2013

Related U.S. Application Data
(63) Continuation of application No. PCT/JP2012/073912, filed on Sep. 19, 2012.

(30) Foreign Application Priority Data
Sep. 30, 2011 (JP) 2011-218014

(51) Int. Cl. C12M 1/00

(52) U.S. Cl. CPC C12M 21/00
USPC 435/303.1; 435/283.1

(57) ABSTRACT

An incubator includes: a mount table having placed thereon a container, a holding member holding the container, or a storage member storing the holding members in a vertical-multi-stage manner; a carrying-in door on one side-face of the incubator including first and second antennas in an interior; a discrimination unit to determine that the container or holding member is placed on the table if radio waves are transmitted/received between the first antenna and a tag of the container or holding member, and determine that the storage member is placed if radio waves are transmitted/received between the second antenna and the tag of the holding member in the storage member; and a warning unit to warn if the container or holding member is placed thereon and a specified storage destination is a place for storage member, or the storage member is placed and the destination is a place for holding member.

### CROSS-REFERENCE TO RELATED APPLICATIONS

This application is a continuation under 35 U.S.C. §120 of PCT/JP2012/073912, filed Sep. 19, 2012, which is incorporated herein reference and which claimed priority to Japanese Application No. 2011-218014, filed Sep. 30, 2011. The present application likewise claims priority under 35 U.S.C. §119 to Japanese Application No 2011-218014, filed Sep. 30, 2011, the entire content of which is also incorporated herein by reference.

### BACKGROUND OF THE INVENTION

1. Field of the Invention

The present invention relates to an incubator and a carrier system.

2. Description of the Related Art

A culture apparatus (incubator) is known as a device for efficiently incubating cultures such as cells while controlling temperature, humidity, for example, as appropriate. In general, a rack called a stacker, which is capable of storing a plurality of containers containing cultures, is stored on a shelf of a culture chamber in the culture apparatus. A carrier device stores the container storing cultures in a specified storage destination in the stacker (see International Publication No. 2007/001002, for example).

As compared with carrying the container with the carrier device, carrying the stacker storing a plurality of containers with the carrier device allows a greater amount of cultures to be efficiently carried into the culture chamber. However, if a user mistakenly specifies the storage destination of the container when causing the carrier device to carry the stacker, the carried stacker might collide with another stacker or a shelf in the culture chamber.

### SUMMARY OF THE INVENTION

An incubator according to an aspect of the present invention, which is configured to automatically carry a container to a specified storage destination and culture a cell or a microbe in the container at the storage destination, the incubator includes: a mount table configured to have placed thereon the container with a tag; a holding member with the tag at the same height as the height of the tag of the container, the holding member configured to hold the container; or a storage member configured to store the holding members in a multi-stage manner in a vertical direction so that the tag is positioned at a height different from the height of the tag of the container or the holding member; a carrying-in door for carrying the container, the holding member, or the storage member into the incubator, the carrying-in door provided on one side face of the incubator, the carrying-in door including in an interior thereof a first antenna disposed at the same height as the height of the tag of the container or the holding member; and a second antenna disposed at the same height as the height of the tag of the holding member stored in the storage member; a discrimination unit configured to, in a state that the container, the holding member, or the storage member is placed on the mount table and the carrying-in door is open, determine that the container or the holding member is placed on the mount table if radio waves can be transmitted/received between the first antenna and the tag of the container or the holding member, with the first antenna and the tag of the container or the holding member being arranged so as to be opposed to each other, and determine that the storage member is placed on the mount table if radio waves can be transmitted/received between the second antenna and the tag of the holding member stored in the storage member, with the second antenna and the tag of the holding member stored in the storage member being arranged so as to be opposed to each other; and a warning unit configured to give warning if the discrimination unit determines that the container or the holding member is placed on the mount table and the specified storage destination is a place for storing the storage member, or if the discrimination unit determines that the storage member is placed on the mount table and the specified storage destination is a place for storing the holding member.

Other features of the present invention will become apparent from descriptions of this specification and of the accompanying drawings.

### BRIEF DESCRIPTION OF DRAWINGS

FIG. 1 is a diagram illustrating a configuration of a carrier system 10 according to an embodiment of the present invention;

FIG. 2 is a front view of an incubator 11;

FIG. 3 is a perspective view of the incubator 11 seen from an +X direction;

FIG. 4 is a front view of the incubator 11 in a state where a carrying in/out door 27 is open;

FIG. 5 is an explanatory view for describing a position of an antenna 14 in the carrying in/out door 27;

FIG. 6 is a perspective view of an incubator unit 30;

FIG. 7 is a diagram illustrating an example of a stacker 200;

FIG. 8 is a diagram illustrating an example of a container 100 and a tray 110;

FIG. 9 is a diagram illustrating compartments of a storage space of a storage rack 41;

FIG. 10 is a diagram illustrating compartments of a storage space of a storage rack 42;

FIG. 11 is a diagram illustrating a configuration of a carrier table 12 and a carrier device 16;

FIG. 12 is a diagram illustrating a state where a stacker 200 is placed on a carrier table 12;

FIG. 13 is an explanatory view for describing a positional relationship between a stacker 200 placed on the carrier table 12 and an antenna 14 provided in a carrying in/out door 27;

FIG. 14 is an explanatory view for describing a positional relationship between a tray 110 placed on a carrier table 12 and an antenna 14 provided in a carrying in/out door 27;

FIG. 15 a diagram illustrating an outline of a configuration of the incubator 11 and a control device 15;

FIG. 16 is an explanatory view for describing reference data to be stored in a storage device 91;

FIG. 17 is a diagram illustrating functional blocks realized by a microcomputer 92;

FIG. 18 is a diagram illustrating an example of a screen displayed on an operation panel 90;

FIG. 19 is a flowchart illustrating an example of a process to be executed when the storage destination is specified;

FIG. 20 is a flowchart illustrating an example of a process to be executed when a carrying target is carried in;

FIG. 21 is an explanatory view for describing a positional relationship between a stacker 600 placed on the carrier table 12 and an antenna 14 provided in a carrying in/out door 610;

FIG. 22 is an explanatory view for describing a positional relationship between the tray 110 placed on the carrier table 12 and an antenna 14 provided in a carrying in/out door 610;

FIG. 23 is an explanatory view for describing a positional relationship between a stacker 650 placed on a carrier table 12 and an antenna 14 provided in a carrying in/out door 27;

FIG. 24 is an explanatory view for describing a positional relationship between a culture flask 700 placed on a carrier table 12 and an antenna 14 provided in a carrying in/out door 27;

FIG. 25 is a diagram illustrating a culture flask 700 seen from a +Z direction;

FIG. 26 is an explanatory view for describing a culture flask 700;

FIG. 27 is an explanatory view for describing a switch 800 provided on a slide plate 67;

FIG. 28 is an explanatory view for describing a stacker 201;

FIG. 29 is an explanatory view for describing an opening portion 311 provided in a stacker 201;

FIG. 30 is a perspective view of a stacker 201;

FIG. 31 is an explanatory view for describing a stacker 202 used when a distance d is great; and

FIG. 32 is an explanatory view for describing an opening portion 312 provided in the stacker 202.

### DETAILED DESCRIPTION OF THE INVENTION

At least the following details will become apparent from descriptions of this specification and of the accompanying drawings.

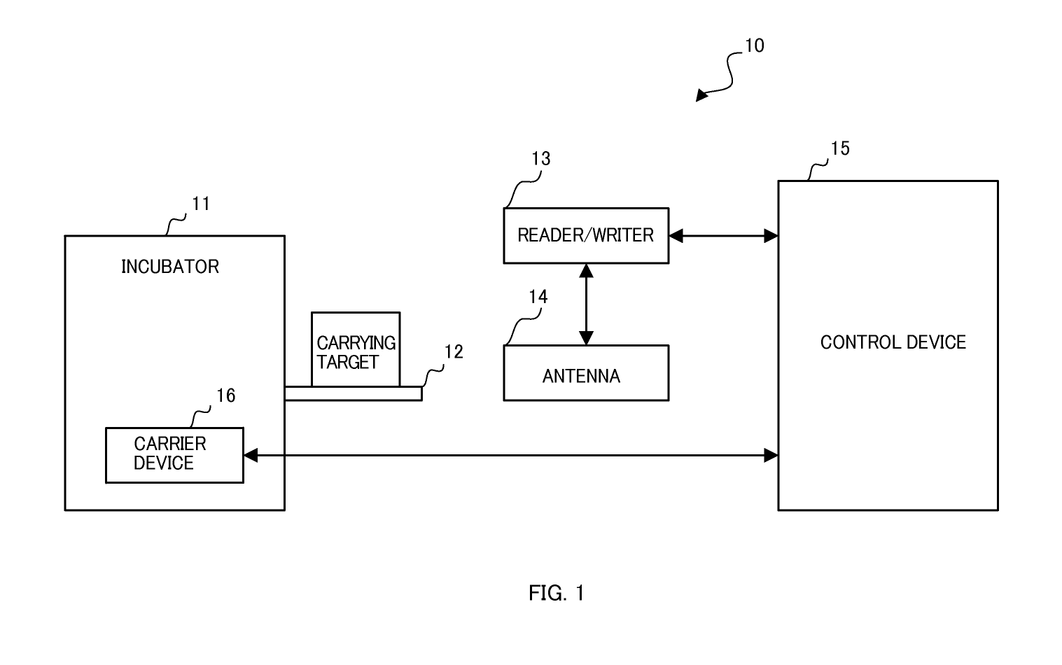

FIG. 1 is a schematic view of a carrying (transport) system 10 according to an embodiment of the present invention. The carrier system 10 is configured to determine the type of a carrying target (for example, a container or a stacker) to be carried to an incubator 11, and carry the carrying target according to the determination result.

The carrier system 10 includes a carrier table 12, a reader/writer 13, an antenna 14, a control device 15, and a carrier device 16.

On the carrier table 12 (mount table), placed is the carrying target, such as a stacker, to be carried into the incubator 11 or the carrying target carried out of the incubator 11.

The reader/writer 13 is configured to read information recorded in a wireless tag (not shown) attached to the carrying target using the antenna 14. The reader/writer 13 is also configured to write information in the wireless tag using the antenna 14. The wireless tag is a storage element also referred to as an IC (Integrated Circuit) tag or RFID (Radio Frequency Identification), and information can be read therefrom and written thereinto via radio waves.

The control device 15 is configured to integrally control, for example, the incubator 11 and the carrier device 16. As will be described later in detail, the control device 15 is configured to control, for example, a temperature control device (not shown) and a humidity control device (not shown) for the incubator 11, thereby adjusting the temperature and humidity of the incubator 11. Further, the control device 15 is configured to determine the type of the carrying target (container or stacker) based on the information read by the reader/writer 13, and control the carrier device 16 according to the determination result.

### ==Outline of Incubator 11==

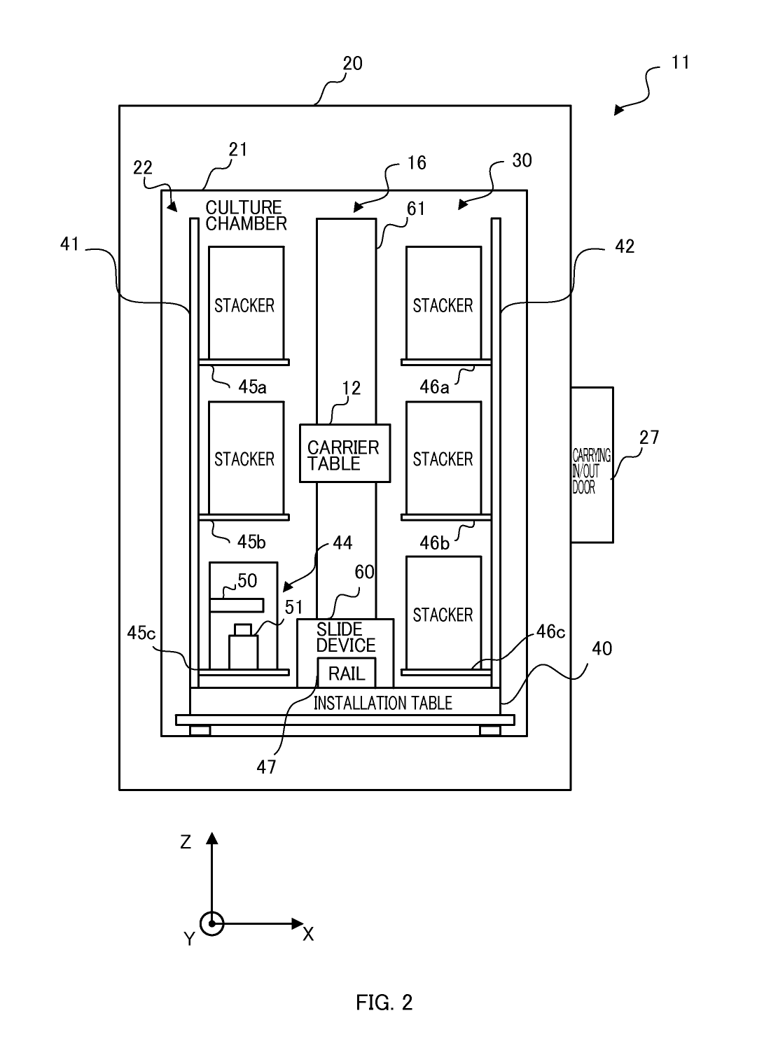

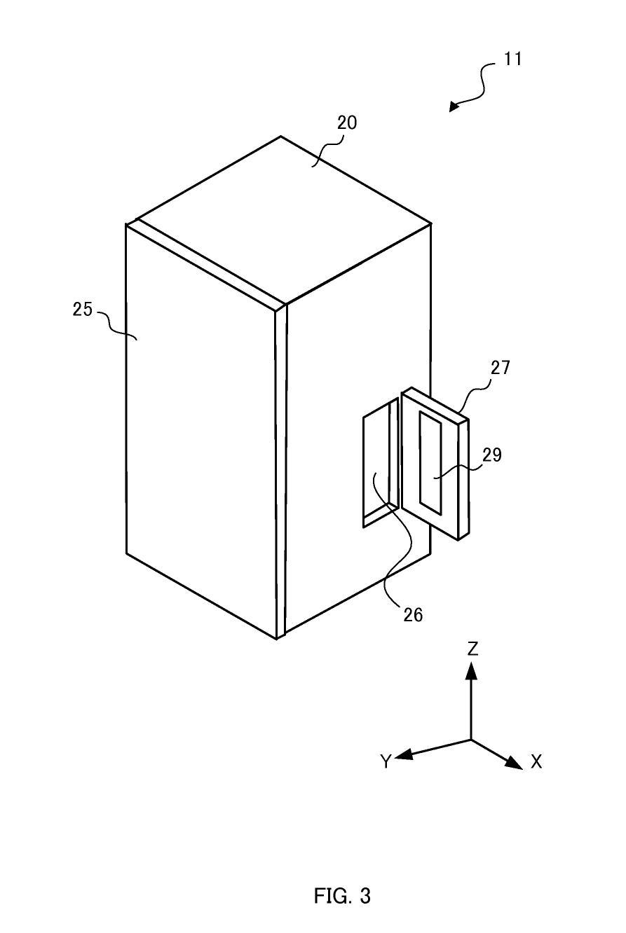

An example of the incubator 11 will be described here with reference to FIGS. 2 and 3. FIG. 2 is a front view of the incubator 11 with a door on the front face (+Y side) being open, and FIG. 3 is an external perspective view of the incubator 11. In an embodiment of the present invention, some components constituting the incubator 10 are appropriately omitted from the drawings so as to facilitate the understanding of the structure of the incubator 10. Further, it is assumed here that, an X-axis direction is the left-right direction with respect to the incubator 11, a Y-axis direction is the front-back direction with respect to the incubator 11, and a Z-axis direction is the up-down direction with respect to the incubator 11.

The incubator 11 is a device configured to incubate a culture such as a cell (specimen) and a microbe, and includes an outer case 20 and an inner case 21.

The outer case 20 is a so-called housing of the incubator 11, and is in a substantially rectangular parallelepiped shape with an opening portion in the front thereof. The inner case 21 having a shape with an opening portion in the front similarly to the outer case 20 is provided in the interior of the outer case 20 so as to be covered with the outer case 20. A front door 25 to open or close the opening portion of the inner case 21 is provided on the front face of the outer case 20. The internal space created in the inner case 21 when the front door 25 is closed is served as a culture chamber 22.

As depicted in FIG. 3, a carrying in/out port 26 penetrating from the outside into the culture chamber 22 is provided on the right side face of the outer case 20 of the incubator 11.

The carrying in/out port 26 (carrying-in port) is an opening portion formed to carry the container storing a culture or the stacker storing the container therethrough into the culture chamber 22, or carry it out of the culture chamber 22. On the right side face of the outer case 20, a carrying in/out door 27 (carrying-in door) is provided which is configured to open outward so as to open or close the carrying in/out port 26. The carrying in/out door 27 is configured to pivot about a hinge (support shaft) provided in the vertical direction, and open outward at an angle of 90° with respect to the opening face of the carrying in/out port 26.

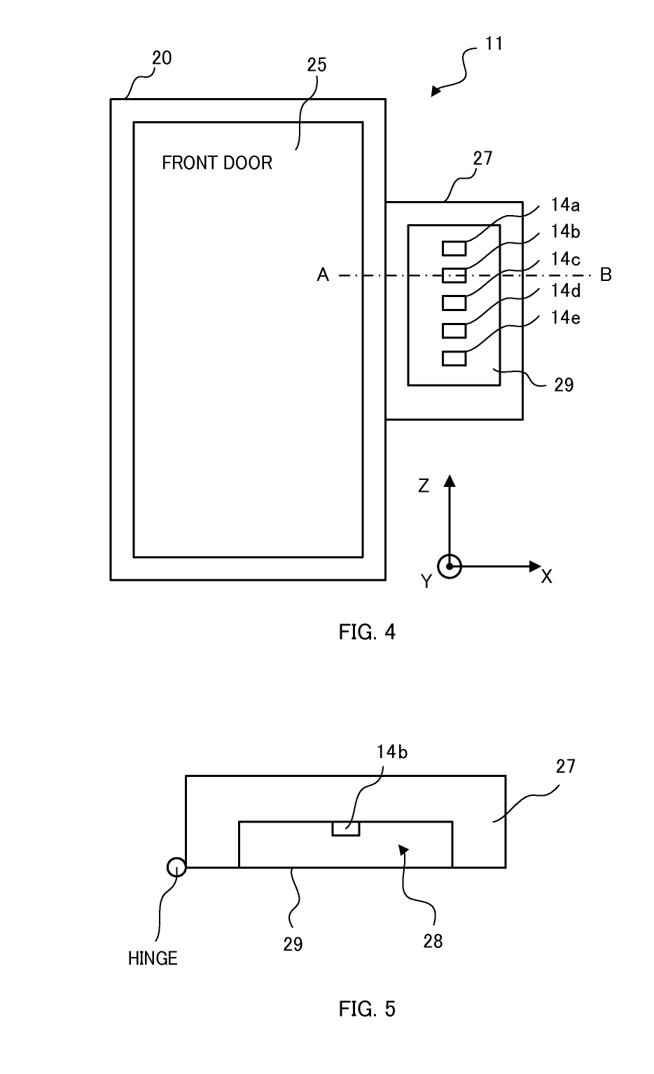

FIG. 4 is a front view of the incubator 11 in a state where the front door 25 is closed and the carrying in/out door 27 is opened at an angle of 90°. FIG. 5 is a cross-sectional diagram of a section A-B of FIG. 4 when seen in the −Z direction.

In a recess 28 formed in an inner surface of the carrying in/out door 27, antennas 14a to 14e are provided which are configured to transmit/receive radio waves to/from the wireless tags attached to, for example, a culture container. The space formed by the recess 28 is hermetically closed with an acrylic plate 29 which allows radio waves to pass therethrough. Therefore, even though the culture chamber 22 is humidified with the carrying in/out door 27 being closed, the antennas 14a to 14e can be prevented from deteriorating due to humidity.

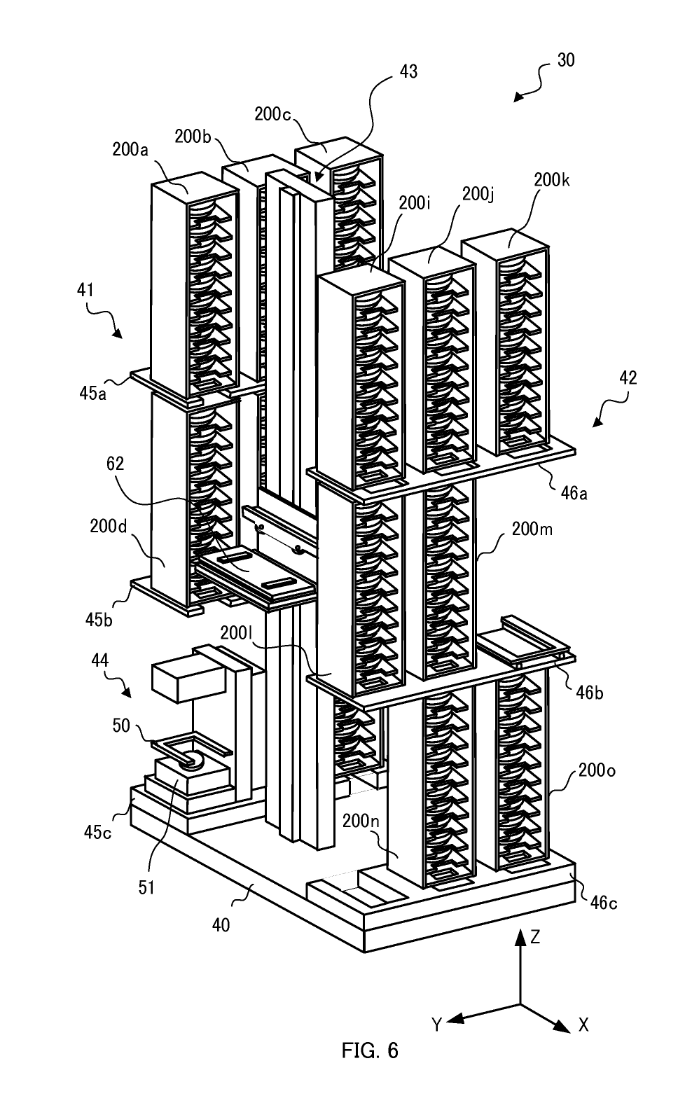

The inner case 21 includes, as depicted in FIG. 2, an incubator unit 30 configured to store the stacker, for example, and the incubator unit 30 includes the carrier table 12, the carrier device 16, an installation stand 40, storage racks 41 and 42, and an observation device 44. FIG. 6 is a perspective view of the incubator unit 30. On the installation stand 40 placed on the bottom of the inner case 21, the storage rack 41 including shelves 45a to 45c, the storage rack 42 including shelves 46a to 46c, and the carrier device 16 are installed. Note that, in FIG. 6, some of the components constituting the incubator unit 30 are appropriately omitted from FIG. 6 to facilitate the understanding of the relationship among the components of the incubator unit 30. The stacker 200 depicted in FIG. 6 stores ten containers for storing culture.

The carrier table 12 has the container or the stacker placed thereon, and the container or the stacker is carried to the specified place by the carrier device 16.

The carrier device 16 is attached so as to be movable on a rail 47 that is attached in the Y-axis direction on the surface of the installation stand 40. The rail 47 is attached at the position between the storage rack 41 and the storage rack 42.

The observation device 44 is a device configured to observe the culture stored in the container, and includes an observation stand 50 and a camera 51.

On the observation stand 50, the container having been carried by the carrier device 16 is placed. The observation stand 50 is provided with motors to move the observation stand 50 in the X-axis, Y-axis, and Z-axis directions, respectively, so that shooting of the container 100 by the camera 51 can be facilitated. The camera 51 is configured to shoot a video or an image of the culture in the container.

### ==Details of Container 100, Tray 110, and Stacker 200==

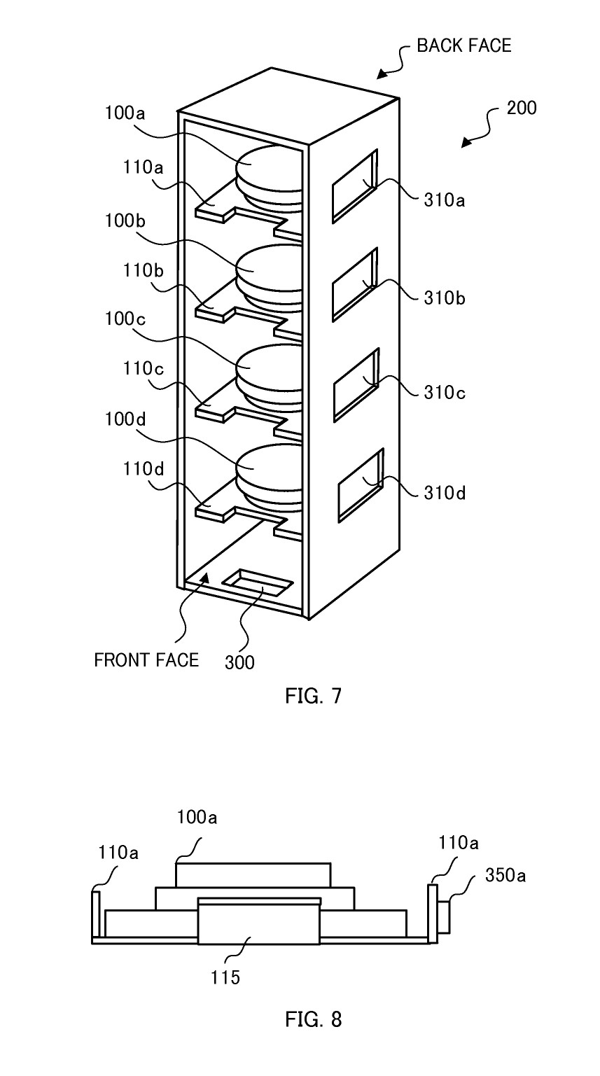

FIG. 7 depicts an example of the stacker 200 (storage member). The stacker 200 is a case in a substantially rectangular parallelepiped shape having opening portions on the front and back faces thereof. The stacker 200 stores, for example, four trays 110a to 110d in a multi-stage manner in the vertical direction. In FIG. 6, the stacker 200 stores ten containers, however, in an embodiment of the present invention, a description will be given assuming that the stacker 200 stores four trays for convenience' sake.

The right side face of the stacker 200 is provided with opening portions 310a to 310d for allowing radio waves to pass therethrough from the antennas 14a to 14d, respectively. The stacker 200 according to an embodiment of the present invention is formed using metal plates. Since the metal has a radio wave shielding property, only the opening portions 310a to 310d in the right side face of the stacker 200 can allow radio waves to transmit therethrough. Note that the material of the stacker 200 is not limited to metal, and any material with a radio wave shielding property may be used. For example, a resin material coated with a coating agent for shielding against radio waves (radio wave shielding) may be used.

The trays 110a to 110d (holding members) are members for holding containers 100a to 100d for storing cultures (including liquid such as culture media). FIG. 8 is a diagram when the tray 110a stored in the stacker 200 is seen from the front (front face of the stacker 200). A writable wireless tag 350a is attached on the right side face (face on the opening portion 310a side) out of the faces extending in the vertical direction at the both ends of the tray 110a. Further, in FIG. 8, a handle 115 for allowing a user to hold the tray 110a is attached on the front face of the tray 110a. The trays 110b to 110d are similar to the tray 110a, and wireless tags 350b to 350d, similar to the tag of the tray 110a, are attached to the trays 110b to 110d, respectively. The wireless tags 350a to 350d have recorded therein identification information capable of uniquely identifying each of the containers 100a to 100d. Note that the tray 110 will be described with the reference numeral of the handle 115 being omitted for convenience's sake.

Two apertures are arranged at the bottom of the stacker 200 (only the aperture 300 is depicted). The apertures are used for positioning the stacker 200 and restraining the horizontal movement of the stacker 200 when the stacker 200 is placed on the carrier table 62. The trays 110a to 110d are also formed with recesses for positioning the trays 110a to 110d and restraining the horizontal movement of the trays 110a to 110d when the trays 110a to 110d each are placed on the carrier table 12.

### ==Storage Compartments (Storage Locations) of Stacker 200==

The storage rack 41 depicted in FIG. 1 and FIG. 6 is a shelf for storing the observation device 44 or a plurality of stackers 200, and the storage rack 41 is installed on the installation stand 40 so as to be arranged near a wall surface on the left side of the inner case 21. The storage rack 41 is provided with three shelves 45a to 45c in the vertical direction (Z-axis direction).

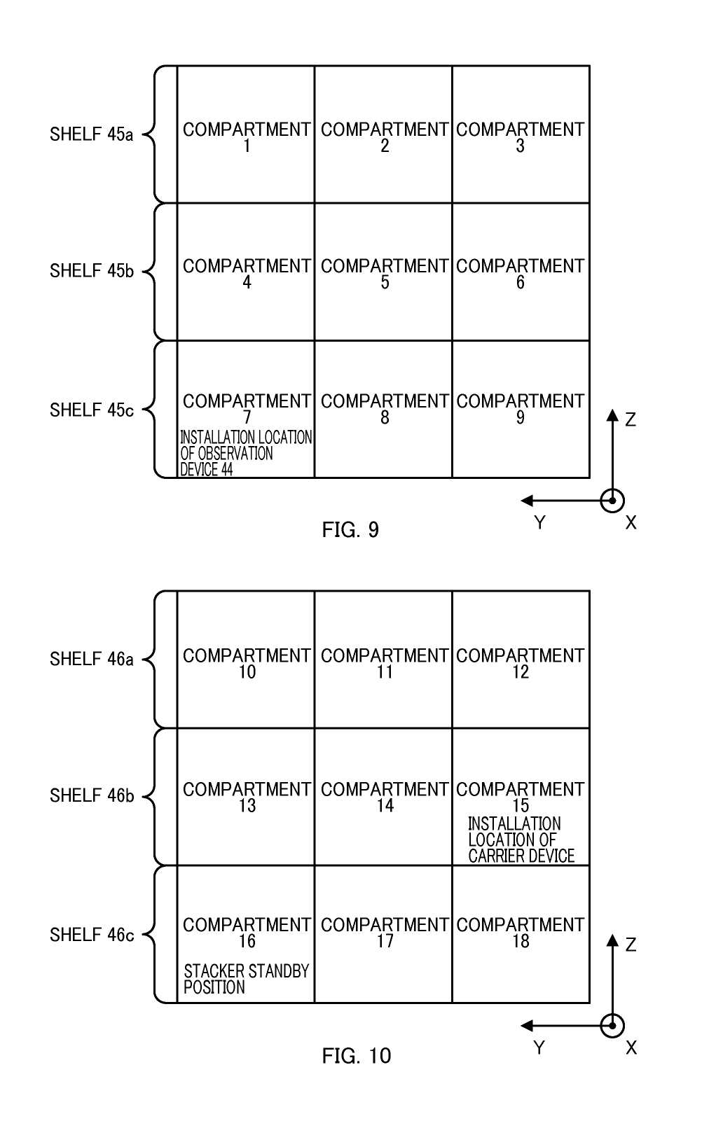

In an embodiment of the present invention, the storage space of the storage rack 41 is sectioned into nine virtual compartments constituted by compartments 1 to 9, as depicted in FIG. 9. Each compartment has a capacity enough to store the stacker 200 or the observation device 44. The storage space of the shelf 45a is allocated to three compartments of the compartments 1 to 3; the storing space of the shelf 45b is allocated to three compartments of the compartments 4 to 6; and the storing space of the shelf 45c is allocated to three compartments of the compartments 7 to 9. The compartment 7 on the front door 25 side of the shelf 45c is a compartment in which the observation device 44 is installed. The tray 110 holding the container 100, which contains the culture to be observed, is set in the observation device 44 during the observation of the culture.

The storage rack 42 is a rack to store a plurality of stackers 200 similarly to the storage rack 41, and is installed on the installation stand 40 so as to be arranged near a wall surface on the right side of the inner case 21. The storage rack 42 is also provided with three shelves 46a to 46c in the vertical direction. The shelves 45a to 45c are attached at heights equal to those at which the shelves 46a to 46c are attached.

As depicted in FIG. 10, the storing space of the storage rack 42 is also sectioned into nine virtual compartments constituted by compartments 10 to 18, similarly to the storage rack 41. The storing space of the shelf 46a is allocated to three compartments of the compartments 10 to 12; the storing space of the shelf 46b is allocated to three compartments of the compartments 13 to 15; and the storing space of the shelf 46c is allocated to three compartments of the compartments 16 to 18. The compartment 15, which is opposed to the carrying in/out port 26 for carrying the stackers 200 into or out of the incubator 11 and is the closest to the carrying in/out port 26, is the compartment for the stacker 200 to be temporarily set therein when the stacker 200 is carried in or out. The compartment 16, which is opposed to the compartment 7 having the observation device 44 set therein, is the compartment for the stacker 200 storing the container 100 containing the culture to temporarily stand by, when the culture is observed with the observation device 44. The stand-by compartment for the stacker 200 to be observed is set to be the compartment 16 opposed to the compartment 7, thereby, for example, being able to reduce the distance for the carrier device 43 to carry the container 100 stored in the stacker 200 in the compartment 16, to the observation device 44.

Although three-stage shelves are provided for each of the storage racks 41 and 42 in an embodiment of the present invention, it is not limited thereto, and for example, only one shelf may be provided instead. In this case, the installation stand 40 may also serve as a shelf.

### ==Details of Carrier Table 12 and Carrier Device 16==

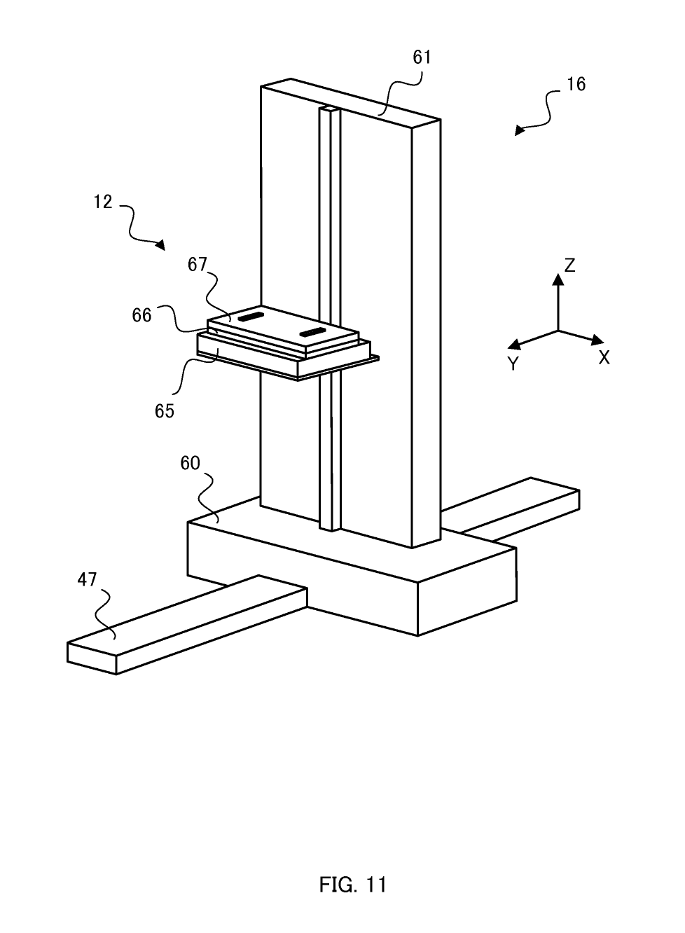

FIG. 11 is a perspective view of the carrier table 12 and the carrier device 16. The carrier device 16 includes a slide device 60 and a rail member 61.

The slide device 60 is attached to a rail 47 in a manner movable along the Y-axis direction. The motor (for Y axis) provided in the interior of the slide device 60 rotates, thereby moving (sliding) the slide device 60 on the rail 47. The slide device 60 is attached with the rail member 61 along the vertical direction.

The carrier table 12 (mount table) includes a base plate 65 and slide plates 66 and 67, and is attached to the rail member 61 in a manner movable along the Z-axis direction. The motor (for Z axis) provided in the interior of the slide device 60 rotates, thereby moving (sliding) the carrier table 12 in the Z-axis direction. The motor (for X axis) provided in the interior of the base plate 65 rotates, thereby sliding the slide plates 66 and 67 in positive and negative directions of the X axis. The tray 110 or the stacker 200 is placed directly on the slide plate 67.

### ==When Stacker 200 is Placed on Carrier Table 12==

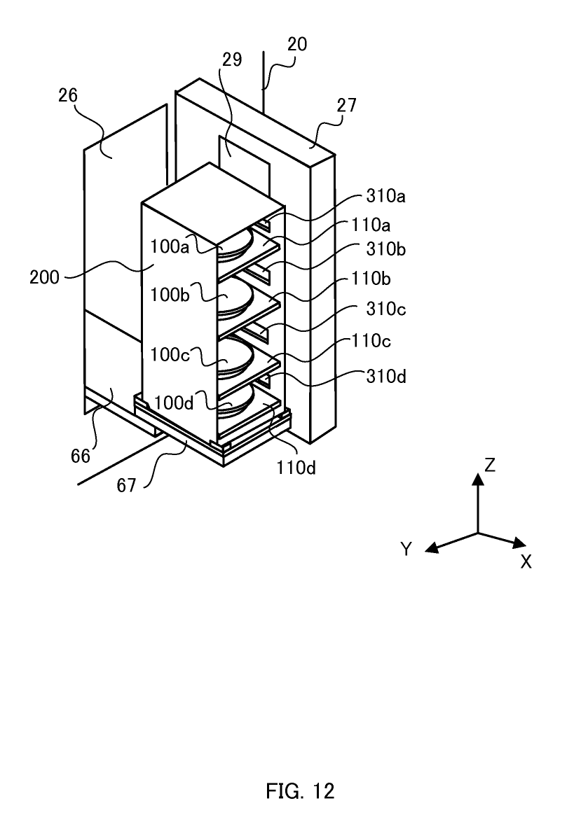

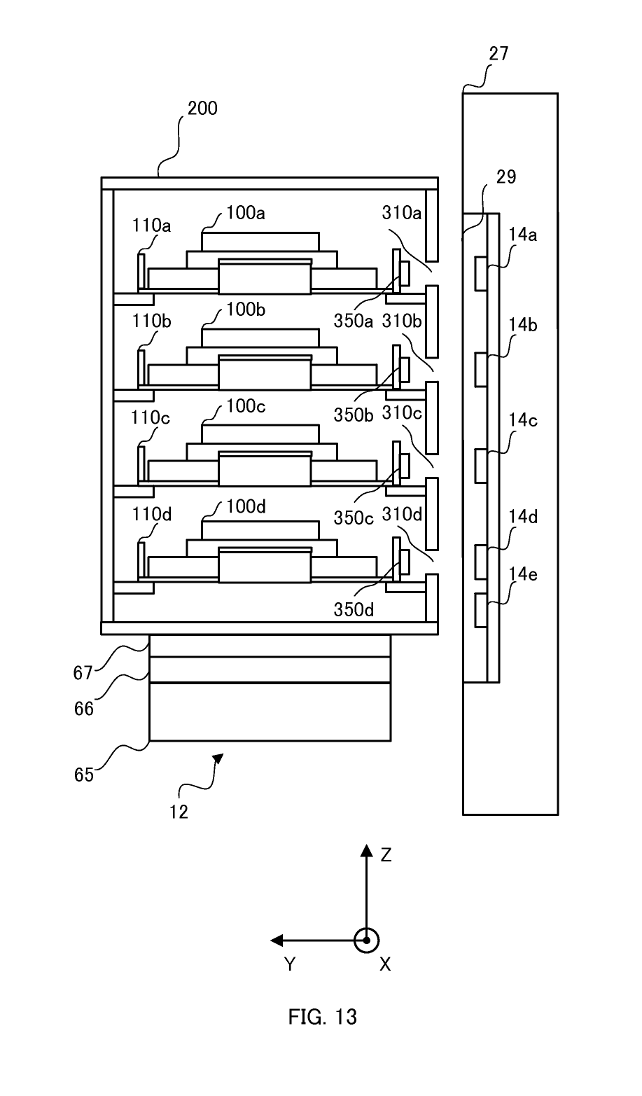

A description will be given here of the state of the stacker 200 placed on the carrier table 12 when a user carries the stacker 200 in, with reference to FIG. 12 and FIG. 13. As depicted in FIG. 12, the slide plates 66 and 67 of the carrier table 12 are slid to the predetermined position (hereinafter, referred to as predetermined position A) at which the stacker 200 can be placed on the slide plate 67. When the stacker 200 is placed in a state where the slide plate 67 has slid to the predetermined position A, the wireless tags 350a to 350d attached to the trays 110a to 110d stored in the stacker 200 are opposed to the antennas 14a to 14d via the opening portions 310a to 310d, as depicted in FIG. 13. As such, since the antennas 14a to 14d are provided at positions corresponding to the positions of the wireless tags 350a to 350d, respectively, the reader/writer 13 depicted in FIG. 1 can read information stored in each of the wireless tags 350a to 350d or write information into the wireless tags 350a to 350d. Whereas, it is impossible to acquire the information of the wireless tag via the antenna 14e. Note that the stacker 200 has a plurality of opening portions 310a to 310d, in order to prevent the crosstalk of radio waves and cause the wireless tags 350a to 350d to correspond to the antennas 14a to 14d, respectively.

### ==When Tray 110a is Placed on Carrier Table 12==

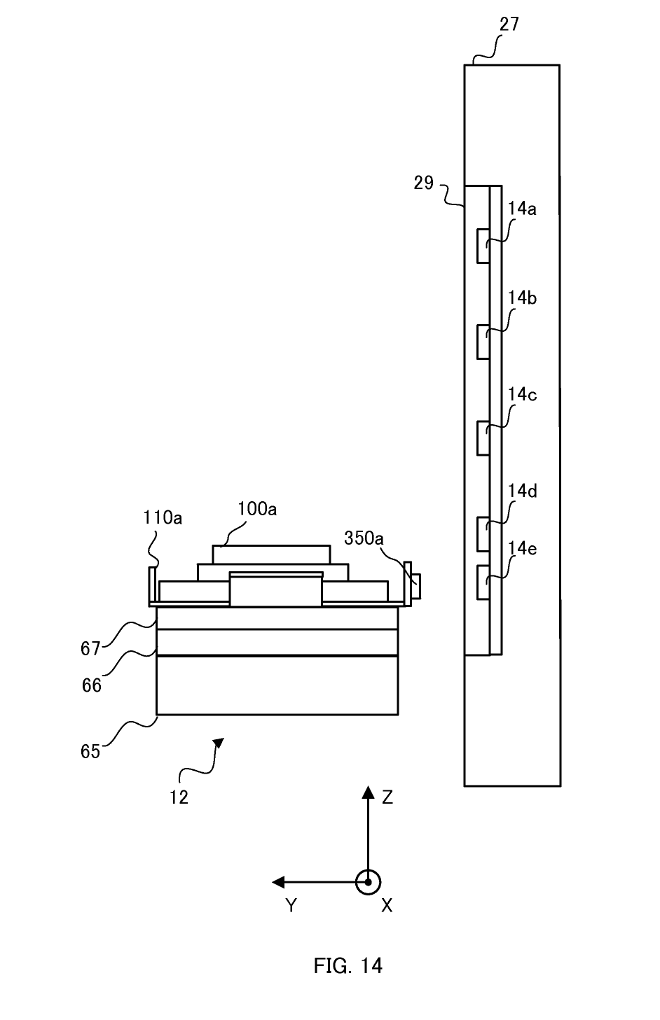

FIG. 14 depicts the state of the tray 110a placed on the carrier table 12 when a user carries the tray 110a in. Note that the slide plates 66 and 67 of the carrier table 12 are also slid to the predetermined position A at which the tray 110a can be placed on the slide plate 67.

When the tray 110a is placed in a state where the slide plate 67 has slid to the predetermined position A, the wireless tag 350a attached to the tray 110a is opposed to the antenna 14e. As such, the antenna 14e is provided at the position corresponding to the position (height) at which the wireless tag 350a is attached to the tray 110a that is directly placed on the slide plate 67, and thus the reader/writer 13 can read the information stored in the wireless tag 350a or writes the information in the wireless tag 350a via the antenna 14e. Note that, on this occasion, it might be possible to read/write the information from/into the wireless tag 350a using the antenna 14d as well.

### <<Summary of Control Device 15 Configured to Integrally Control Incubator 11, for Example>>

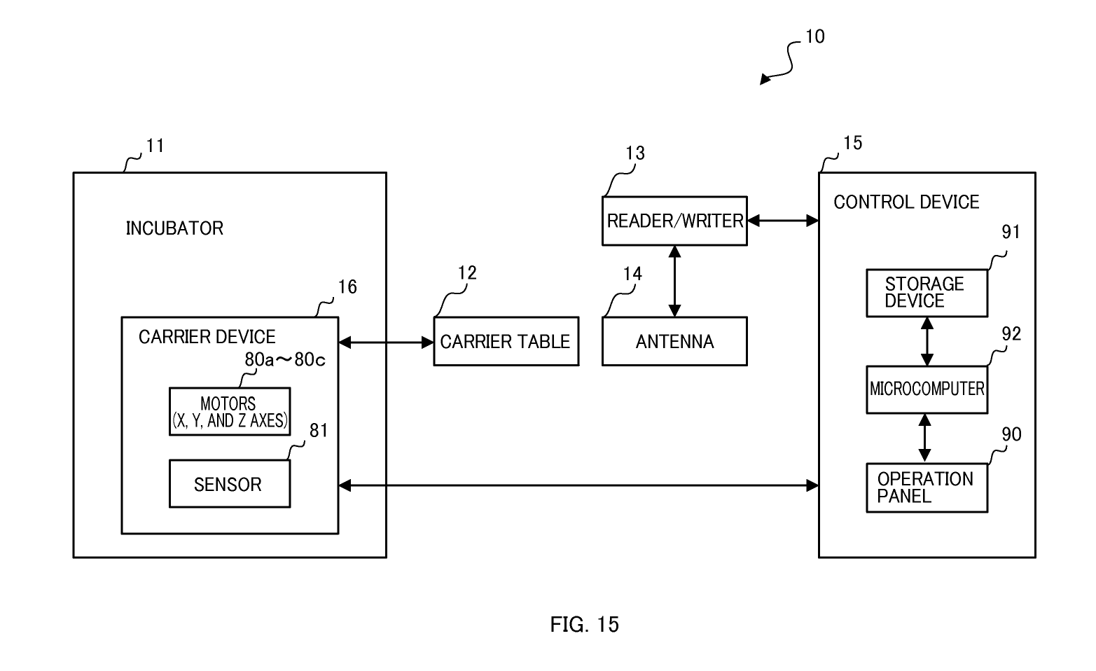

A description will be given here of the control device 15 configured to control, for example, the incubator 11, with reference to FIG. 15. FIG. 15 depicts the main configuration of blocks included in the carrier system 10, and for example, only the main blocks related to the control system in the incubator 11 are depicted while other blocks are omitted. Further, in FIG. 15, the detailed descriptions of the blocks common to those in another drawing will be omitted.

The carrier device 16 of the incubator 11 includes motors 80a to 80c and a sensor 81. The motor 80a is a motor for X-axis for moving the slide plates 66 and 67 of the carrier table 12 in the X-axis direction depicted in FIG. 11. The motor 80b is a motor for Y-axis for moving the slide device 60 in the Y-axis direction, and the motor 80c is a motor for Z-axis for moving the carrier table 12 in the Z-axis direction. The sensor 81 is configured to detect the positional information of each of the slide device 60, the carrier table 12, and the slide plates 66 and 67.

The control device 15 is a device configured to integrally control the carrier system 10 and the incubator 11 (for example, a terminal device such as a personal computer), and includes an operation panel 90, a storage device 91, and a microcomputer 92.

The operation panel 90 (input unit) is a panel (for example, a touch panel) for allowing a user to set the operation of the carrier system 10 and the incubator 11. The results of operations on the operation panel 90 are transmitted to the microcomputer 92, and the control device 15 controls each of the blocks in the incubator 11 based on the results of the operations. Further, the operation panel 90 is configured to display various types of information such as the results of the operations, the state of the incubator 11 (for example, temperature and humidity), and the storage locations of the stacker or the container. The operation panel 90 is provided in the control device 15 (the aforementioned personal computer), however, it is not limited thereto. The operation panel 90 may be provided, for example, on the exterior wall surface of the incubator 11.

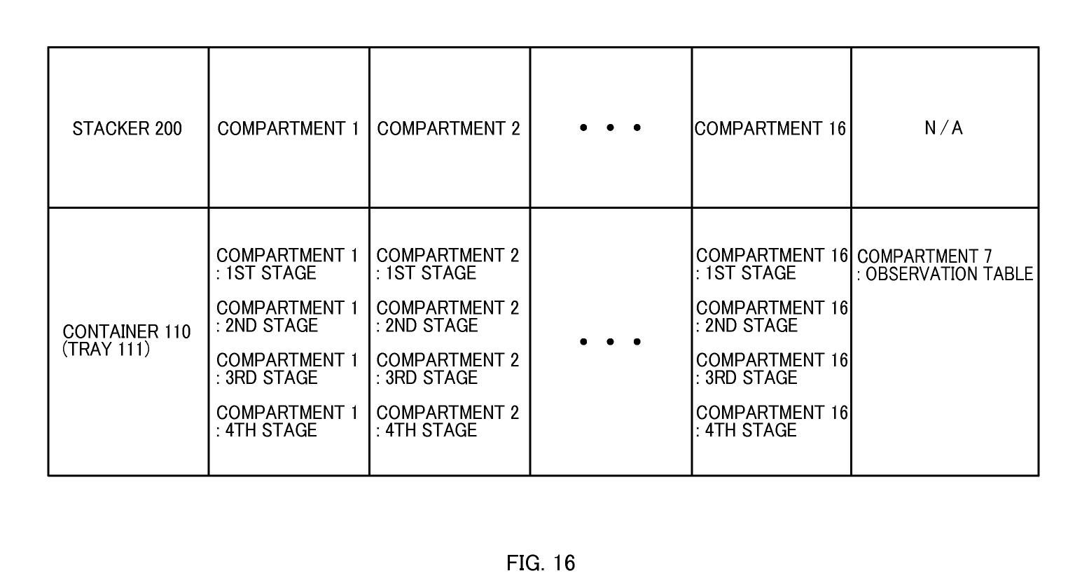

The storage device 91 is configured to store program data to be executed by the microcomputer 92 and various types of data. For example, as depicted in FIG. 16, the storage device 91 stores reference data indicative of the predetermined storage destination at which the stacker 200 can be stored and the predetermined storage destination at which the tray 110 can be stored.

The predetermined storage destinations at which the stacker 200 can be stored are spaces such as “compartment 1”, “compartment 2”, and “compartment 16”. Note that the “compartment 1” and the like refer to the compartments which have been described with reference to FIG. 9 and FIG. 10.

The predetermined storage destinations at which the tray 110 holding the container 110 can be stored are, for example, spaces such as “compartment 1: 1st stage” to “compartment 1: 4th stage” and “compartment 2: 1st stage” to “compartment 2: 4th stage”. Note that “compartment 1: 1st stage” refers to the uppermost space in the spaces of the stacker 200 configured to store the trays 110 when the stacker 200 is stored in “compartment 1”. “Section 1: 4th stage” refers to the lowermost space in the spaces of the stacker 200 configured to store the trays 110 when the stacker 200 is stored in “compartment 1”. “Section 7: observation stand” refers to the space in which the observation stand 50 of the observation device 44 in which the tray 110 is placed.

### ==Functional Blocks of Microcomputer 92==

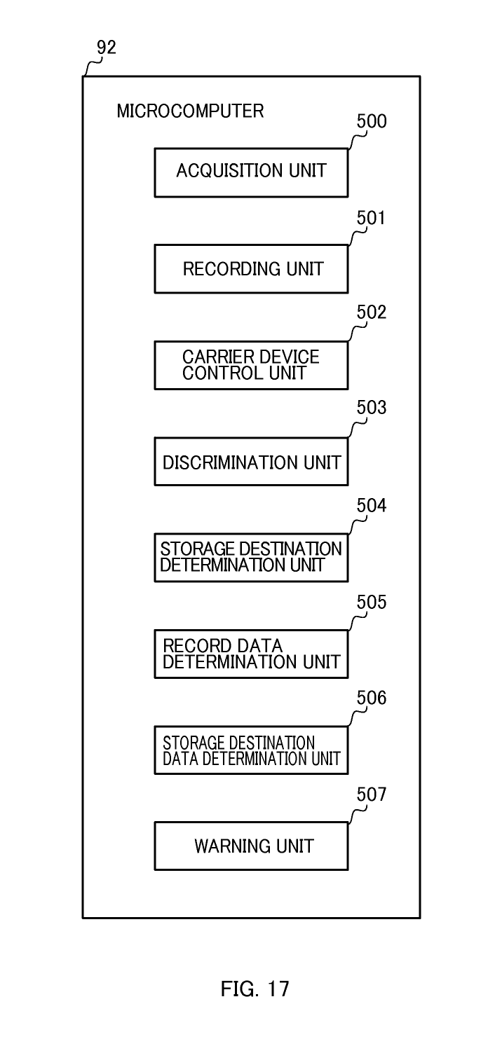

FIG. 17 is a diagram illustrating functional blocks realized by the microcomputer 92 by the microcomputer's executing the program data. In the microcomputer 92, realized are an acquisition unit 500, a recording unit 501, a carrier device control unit 502, a discrimination unit 503, a storage destination determination unit 504, a record data determination unit 505, a storage destination data determination unit 506, and a warning unit 507.

The acquisition unit 500 is configured to cause the reader/writer 13 to read the information of the wireless tag 350, and acquire the information read by the reader/writer 13. In an embodiment of the present invention, if the stacker 200 is placed on the carrier table 12 as depicted in FIG. 13, the acquisition unit 500 acquires the information recorded in the wireless tags 350a to 350d. Whereas, if the tray 110 is placed on the carrier table 12 as depicted in FIG. 14, the acquisition unit 500 acquires the information recorded in the wireless tag 350a.

The recording unit 501 is configured to control the reader/writer 13 so as to record, for example, data indicative of the specified storage destination, information about the culture, into the wireless tag 350. The storage destination data indicative of the storage destination correspond to the data including the information indicative of “compartment” and the information indicative of “stage” or “observation stand”. Note that the information indicative of “stage” or “observation stand” is hereinafter simply referred to as information indicative of “stage”, for convenience's sake.

The carrier device control unit 502 is configured to control the motors 80a to 80c based on, for example, the results of operations of the operation panel 90 (hereinafter, referred to as operation results).

The discrimination unit 503 is capable of determining whether the carrying target (placed object) placed on the carrier table 12 is a container or a stacker, based on the acquisition result of the acquisition unit 500. In an embodiment of the present invention, when the information of the wireless tag is acquired via the antennas 14a to 14d, the discrimination unit 503 determines that the carrying target is the stacker. Whereas, when the information of the wireless tag is acquired via the antenna 14e, the discrimination unit 503 determines that the carrying target is the container. In the aforementioned example of FIG. 14, since the information of the wireless tag is acquired via the antenna 14e, the discrimination unit 503 determines that the carrying target is the container.

The antenna 14e and the control device 15 correspond to one reading device and one writing device, and the antennas 14a to 14d and the control device 15 correspond to a plurality of reading devices and a plurality of writing devices.

The storage destination determination unit 504 is configured to determine whether the storage destination of the carrying target is appropriate or not based on the determination result of the discrimination unit 503, the storage destination of the carrying target specified by a user (first information), and reference data stored in the storage device 91 (second information). Specifically, in the case where the determination result indicates the stacker, if the specified storage destination coincides with any of the predetermined storage destinations of the stacker in the reference data of FIG. 16, the storage destination determination unit 504 determines that the storage destination of the carrying target is appropriate. Further, in the case where the determination result indicates the container, if the specified storage destination coincides with any of the predetermined storage destinations of the container in the reference data, the storage destination determination unit 504 determines that the storage destination of the carrying target is appropriate. Whereas, if the storage destination specified when the determination result indicates the stacker is the storage destination of the container, or if the storage destination specified when the determination result indicates the container is the storage destination of the stacker, the storage destination determination unit 504 determines that the storage destination of the carrying target is not appropriate. The storage destination determination unit 504 corresponds to the determination unit.

The record data determination unit 505 is configured to determine whether or not the wireless tag 350 has recorded therein the information (data) indicative of the storage destination.

The storage destination data determination unit 506 is configured to determine whether or not the specified storage destination is the same as the storage destination recorded in the wireless tag 350. Specifically, if the carrying target is the stacker, the storage destination data determination unit 506 determines whether or not the “compartment” recorded in the wireless tag 350 is the same as the specified “compartment”. If the carrying target is the container, the storage destination data determination unit 506 determines whether or not the “compartment: stage” recorded in the wireless tag 350 is the same as the specified “compartment: stage”. Note that whether or not the “compartments” coincide with each other is determined by comparing the storage destination data indicative of the “compartments”, and whether or not the “compartments: stages” coincide with each other is determined by comparing the storage destination data indicative of the “compartment: stage”.

The warning unit 507 is configured to display warning on the operation panel 90 when it has been determined that the carrying target is not appropriate or when it has been determined that the specified storage destination is different from the storage destination recorded in the wireless tag 350.

### ==Example of Screen Displayed on Operation Panel 90==

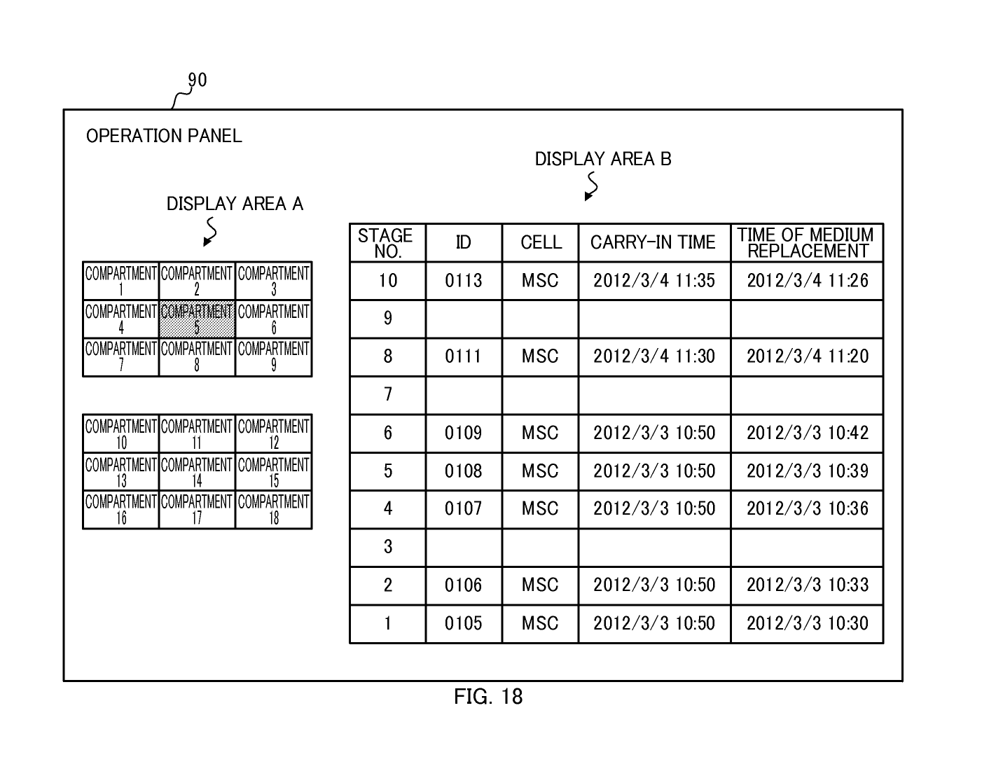

FIG. 18 is a diagram illustrating an example of a screen displayed on the operation panel 90. As depicted in FIG. 18, a display area A of the operation panel 90 displays the compartments in the incubator 11, while a display area B displays the details of the storage objects stored in the compartment that is selected from the compartment 1 to the compartment 18. For example, “compartment 5” is selected here and the selected “compartment 5” stores the stacker 200. Out of 1st to 10th stages of the stacker 200 in the “compartment 5”, 1st, 2nd, 4th to 6th, 8th, and 10th stages stores seven containers, respectively. For example, the container stored in the 1st stage has an ID of “0105”, and the container stores “MSC (Marrow stromal cell)” as a cell. Further, the container stored in the 1st stage was carried into the incubator 11 at 10:50 on Mar. 3, 2012, and the medium of the container was replaced at 10:30 on Mar. 3, 2012. Such information is displayed by reading the information recorded in the wireless tag when, for example, the container is put into the incubator. Note that the detailed descriptions of the containers stored in other stages will be omitted since the containers are similar to that stored in the 1st stage. Further, the stages storing no containers (3rd, 7th, and 9th stages) are displayed as blank spaces, for example. Note that if no stacker is stored in the specified compartment, the display area B displays “no stacker”, for example.

### ==Example of Process for Specifying Storage Destination==

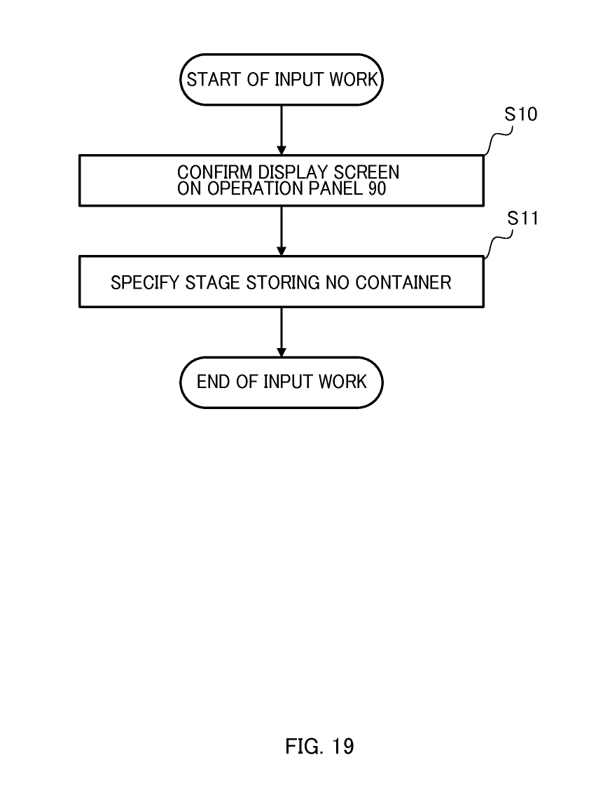

FIG. 19 is a flowchart illustrating an example of a process to be executed for specifying the storage destination. A user causes the operation panel 90 to display the screen depicted in FIG. 18, and confirms the contents on the screen (S10). Then, the user specifies the stage at which the container can be stored in the selected compartment, i.e., the blank stage (S11). Thus, the storage destination of the object to be stored is specified. Since the operation panel 90 is, for example, a touch panel as described above, the user can specify the storage destination by tapping the blank stage.

### ==Example of Process of Carrying Storage Object In==

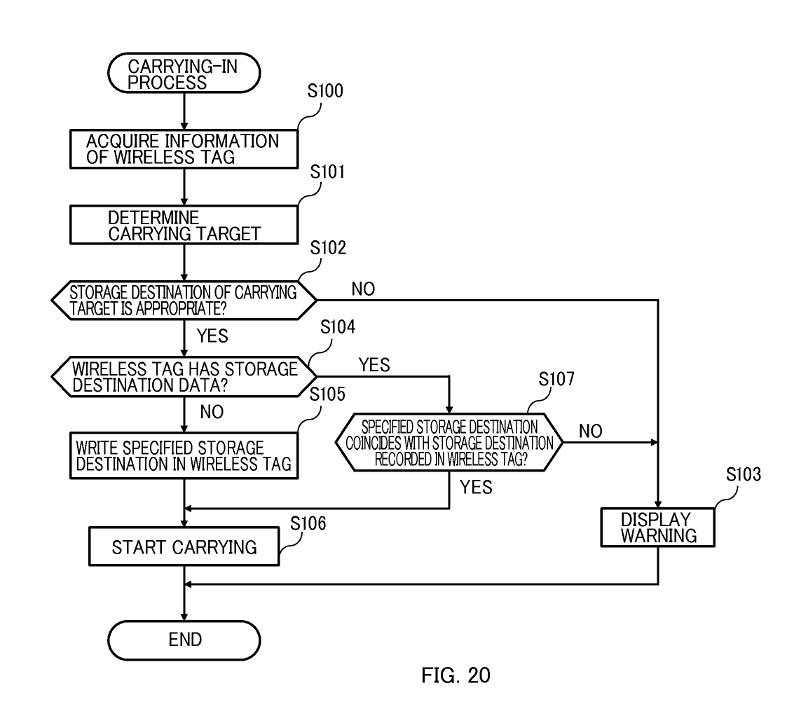

FIG. 20 is a flowchart illustrating one example of a process to be executed for carrying the carrying target (storage object) in. Note here that the user operates the operation panel 90 to specify in advance the storage destination at which the carrying target is to be stored. Further, it is assumed that the carrying target (container 100 or stacker 200) to be stored is placed on the carrier table.

First, when the carrying-in instruction is inputted through the operation panel 90, the acquisition unit 500 acquires the information of the wireless tag of the carrying target read by the reader/writer 13 (S100). The discrimination unit 503 determines whether the carrying target is the container 100 or the stacker 200 based on the information acquired by the acquisition unit 500 (S101). Then, based on the determination result, the specified storage destination, and the reference data, the storage destination determination unit 504 determines whether or not the specified storage destination for the carrying target is appropriate (S102).

If the specified storage destination for the carrying target is not appropriate (S102: NO), that is, for example, if the predetermined storage destination for “container 100” is specified although the carrying target is “stacker 200”, the warning unit 507 displays warning on the operation panel 90 (S103). Whereas, if the specified storage destination for the carrying target is appropriate (S102: YES), the record data determination unit 505 determines whether or not the storage destination data are stored in the wireless tag 350 of the carrying target (S104).

If the wireless tag 350 of the carrying target does not have the storage destination data recorded therein (S104: NO), that is, for example, if the carrying target is carried in the culture chamber 22 for the first time, the recording unit 501 records the specified storage destination data in the wireless tag 350 (S105). Then, when the storage destination data is recorded in the wireless tag 350, the carrier device control unit 502 carries the carrying target to the specified storage destination (S106). Whereas, if the wireless tag 350 of the carrying target has the storage destination data recorded therein (S104:YES), that is, for example, if the carrying target carried out of the chamber once is carried back thereinto again, the storage destination data determination unit 506 determines whether or not the specified storage destination coincides with the storage destination recorded in the wireless tag 350 of the carrying target (S107).

When the storage destination data determination unit 506 determines that the specified storage destination has coincided with the storage destination recorded in the wireless tag 350 of the carrying target (S107: YES), the carrier device control unit 502 carries the carrying target to the specified storage destination (S106). Whereas, when the storage destination data determination unit 506 determines that the specified storage destination has not coincided with the storage destination recorded in the wireless tag 350 of the carrying target (S107: NO), the warning unit 507 displays warning on the operation panel 90 (S103).

### ==Process of Carrying Storage Object Out==

In an embodiment of the present invention, the control unit 92 stores the specified storage destination data in the storage device 91 in such a manner that the specified storage destination data is associated with read identification information of the wireless tag 350. Thus, when the storage destination of the storage object or the identification information is inputted, the carrier device control unit 502 can cause the specified storage object to be carried out based on the information stored in the storage device 91.

==Method of Determining Carrying Target when Stacker has Low Height==

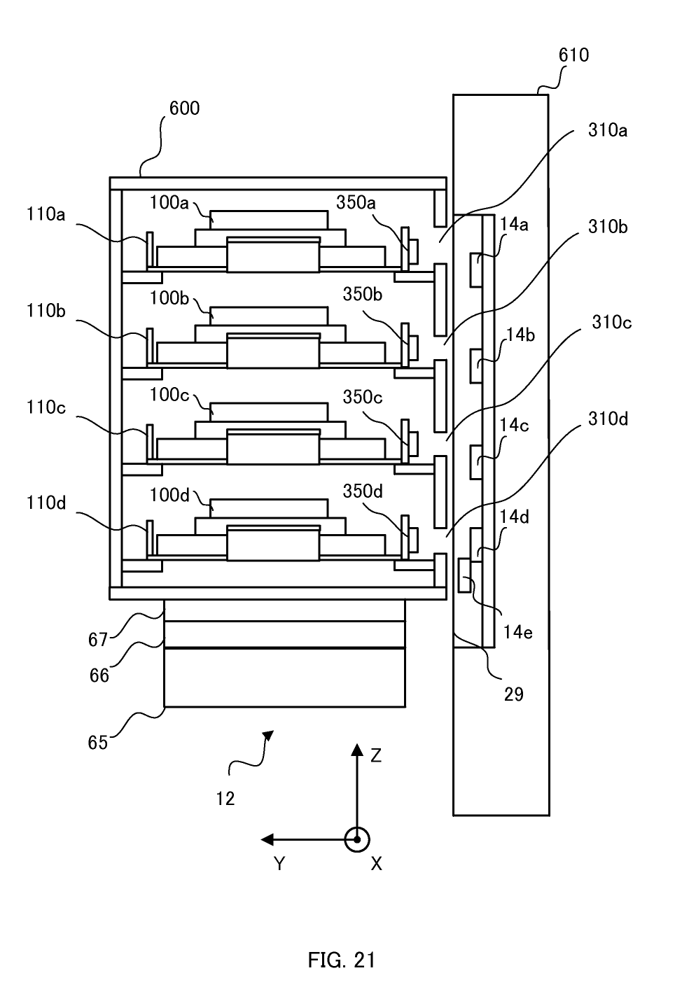

FIG. 21 is an explanatory diagram for describing the positional relationship between a stacker 600 having a lower height than that of the stacker 200, and the antennas 14 in a carrying in/out door 610.

The stacker 600 is similar to the stacker 200 except that, due to the low height of the stacker 600, the antenna 14e (second antenna) is provided covering a part of the antenna 14d (first antenna) which corresponds to the wireless tag 350d. Since the wireless tag 350d is opposed to the antenna 14d via the opening portion 310d, the antenna 14d can transmit/receive radio waves to/from the wireless tag 350d. Whereas, since the antenna 14e is arranged lower than the opening portion 310d (second opening portion), a side face of the stacker 600 shields against radio waves transmitted from the antenna 14e. Thus, the antenna 14e cannot transmit/receive radio waves to/from the wireless tag 350d, and therefore, the information recorded in the wireless tag 350d cannot be read by the antenna 14e.

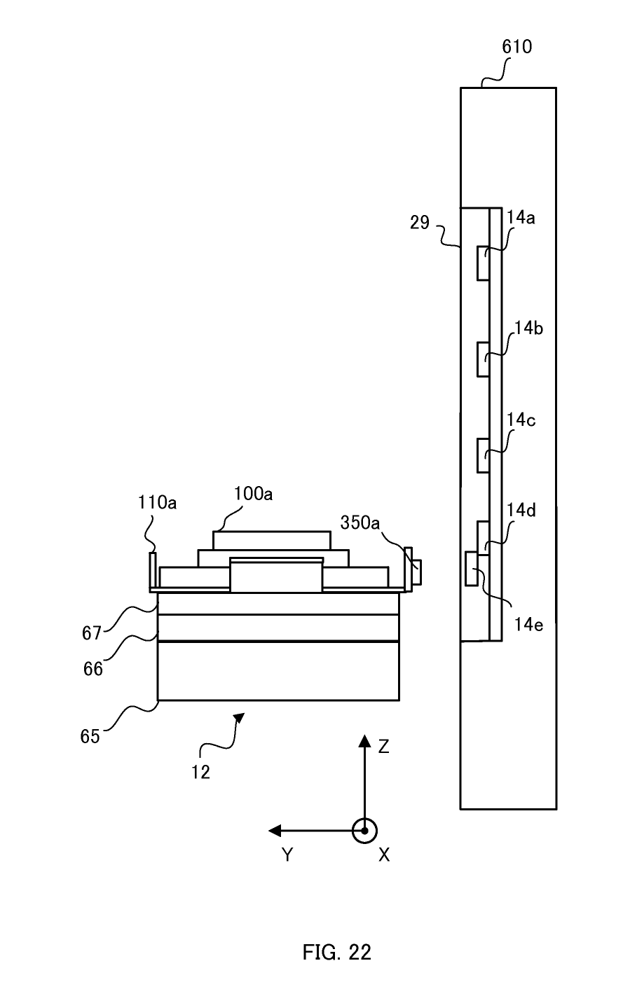

FIG. 22 is a diagram illustrating the positional relationship between the tray 110 and the antenna 14 in the carrying in/out door 610. In this case, both of the antennas 14d and 14e transmits/receives radio waves to/from the wireless tag 350d. Thus, the information recorded in the wireless tag 350d is read by the antennas 14d and 14e. Therefore, in the case where, for example, the stacker 600 is used, it is possible to determine the carrying target, as long as the discrimination unit 503 is allowed to determine that the carrying target is the container only when the information of the wireless tag is acquired via the antenna 14e. Note that the reader/writer 13 corresponds to first and second reading devices, and the control device 15 (discrimination unit 503) corresponds to a discrimination device.

==Method of Determining when Using Wireless Tag Having Information Indicative of Stacker Recorded Therein==

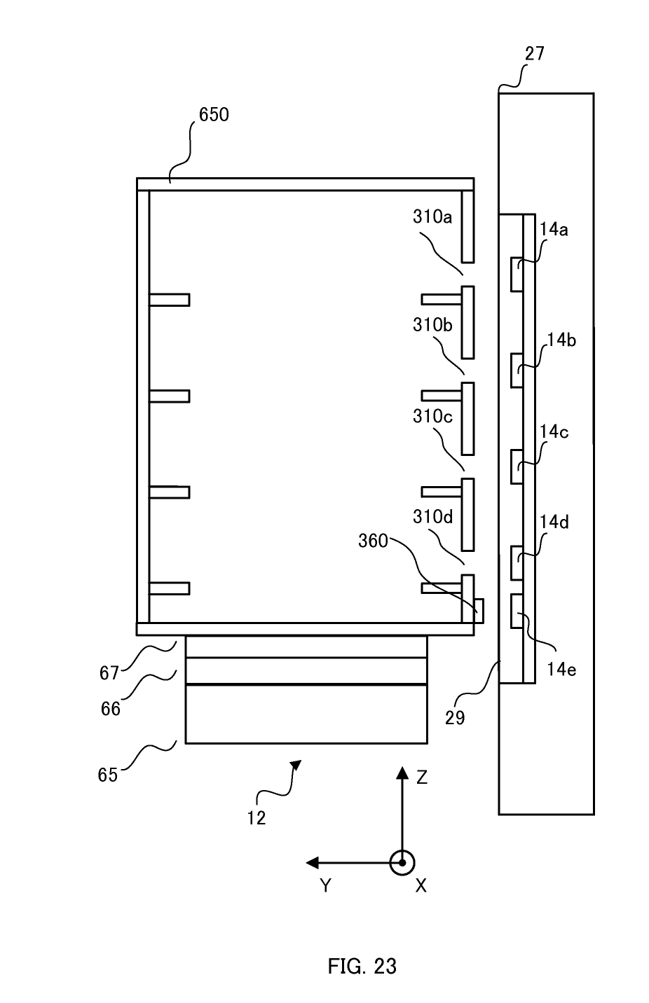

FIG. 23 is a diagram for describing the positional relationship between a stacker 650 and the antennas 14 in the carrying in/out door 27.

The stacker 650 is similar to the stacker 200 except that a wireless tag 360 (second tag), having the information indicative of “stacker” recorded therein, is attached at the position, opposed to the antenna 14e, on a side face below the opening portion 310d of the stacker 650. Thus, as depicted in FIG. 23, when the stacker 650 is placed on the carrier table 12, the antenna 14e receives the information indicative of “stacker” recorded in the wireless tag 350d.

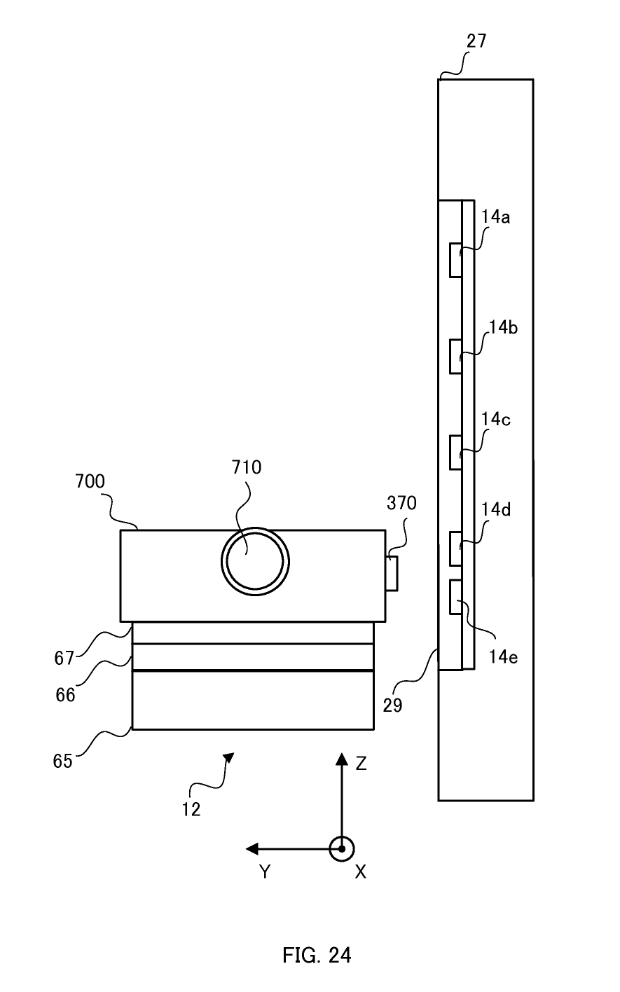

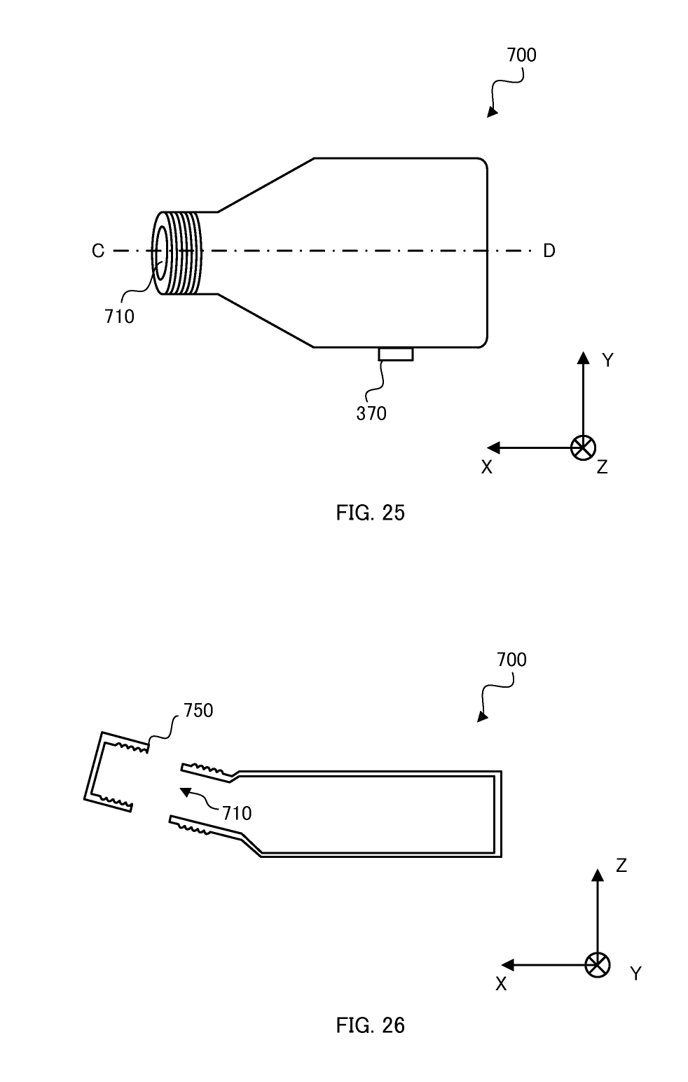

FIG. 24 is a diagram illustrating a state where a culture flask 700 storing a culture is placed on the carrier table 12 in place of the tray 110a. FIG. 25 is a diagram illustrating the culture flask 700 seen from the +Z direction, and FIG. 26 is a cross-sectional diagram along the line C-D of FIG. 25 seen from the +Y direction.

An opening portion 710 for carrying the culture in/out is provided on the front face of the culture flask 700. After the culture is stored in the culture flask 700, the opening portion 710 is closed with a screw cap (stopper member) 750. A wireless tag 370 (first tag), having the information indicative of “container” recorded therein, is provided on the right side face of the culture flask 700 (surface on the carrying in/out door 27 side). In the state where the culture flask 700 is placed on the carrier table 12, the wireless tag 370 transmits/receives radio waves to/from the antennas 14d and 14e. In this case, the antennas 14d and 14e receive the information indicative of “container” recorded in the wireless tag 370. Thus, based on the information indicative of “container” received with the antennas 14d and 14e, the discrimination unit 503 can determine that the carrying target is “container (culture flask)”. Note that, in this case, since the wireless tag 370 has the information indicative of “container” recorded therein, if any one of the antennas 14a to 14e can transmit/receive radio waves to/from the wireless tag 370, it can be determined that the carrying target is “container”. The reader/writer 13 corresponds to a reading device, and the control device 15 (discrimination unit 503) corresponds to a discrimination device.

### ==Other Method of Determining Carrying Target==

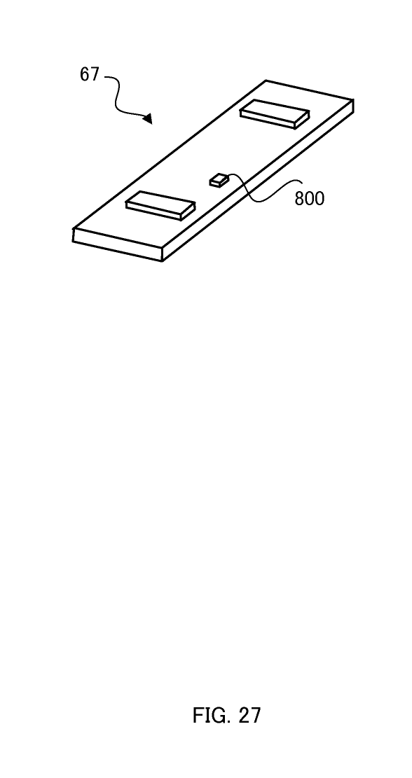

In determining the carrying target, a switch 800 may be provided at a predetermined position of the slide plate 67 on the carrier table 12 as depicted in FIG. 27, for example. Then, the bottom of only the stacker 200 out of the tray 110 and the stacker 200 may have an aperture (not shown) that prevents the switch 800 from being pressed when the stacker 200 is placed on the slide plate 67. In this case, when the tray 110 is placed on the carrier table 12, the switch 800 is pressed and when the stacker 200 is placed thereon, the switch 800 is not pressed. Thus, the carrying target can be determined by causing, for example, the discrimination unit 503 to detect the state of the switch 800.

Since the stacker 200 is heavier than the tray 110, for example, a pressure sensor (not shown) may be used in place of the switch 800, for example, and the carrying target may be determined based on the output of the pressure sensor.

### ==Other Embodiments of Stacker==

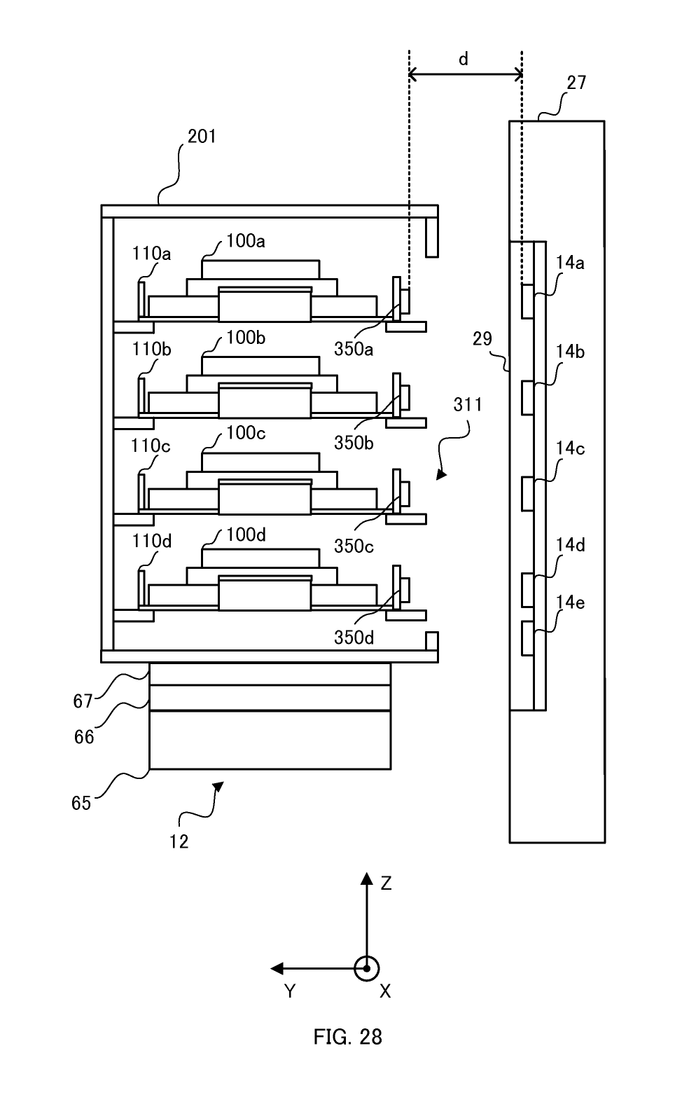

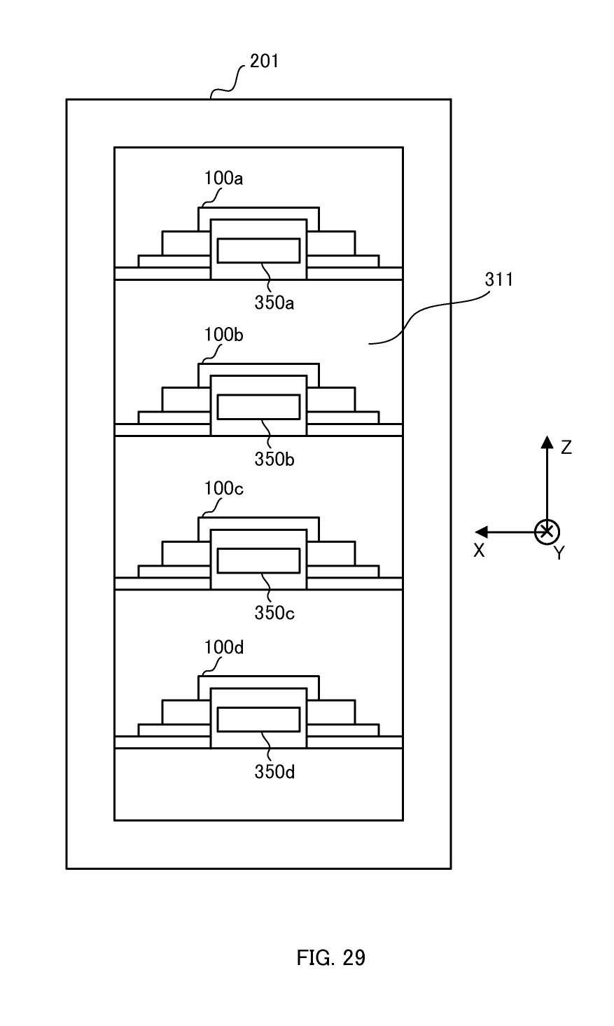

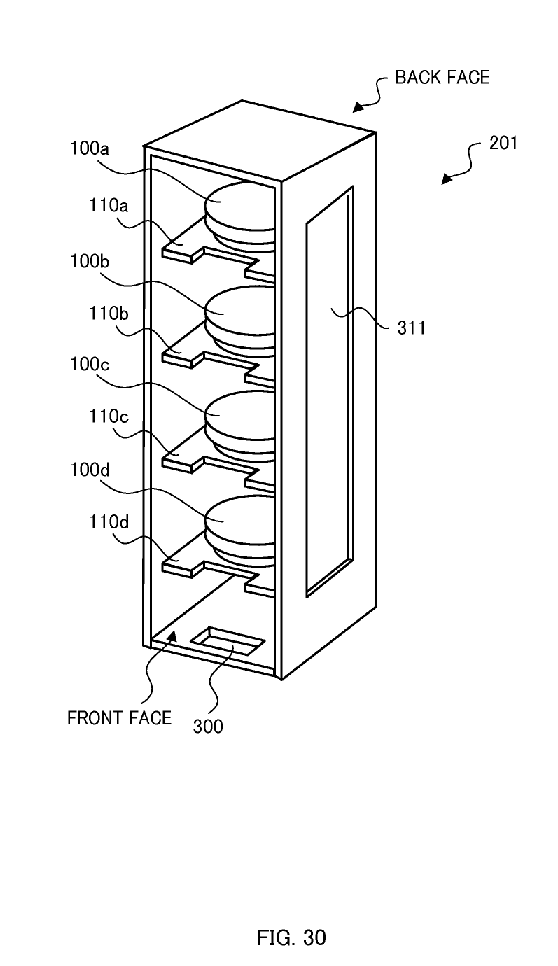

A description will be given here of a stacker 201 used when the antennas 14 has high radio-wave directivity with reference to FIGS. 28 to 30.

As depicted in FIGS. 28 to 30, aside face of the stacker 201 has one opening portion 311. When the antennas 14a to 14e have high radio-wave directivity, crosstalk does not occur in general. Thus, in this case, radio waves can be transmitted/received without crosstalk as long as the wireless tags 350a to 350d and the antennas 14a to 14d are arranged so as to be opposed to each other, respectively, even though one opening portion 311 is arranged with respect to the whole side face of the stacker 201.

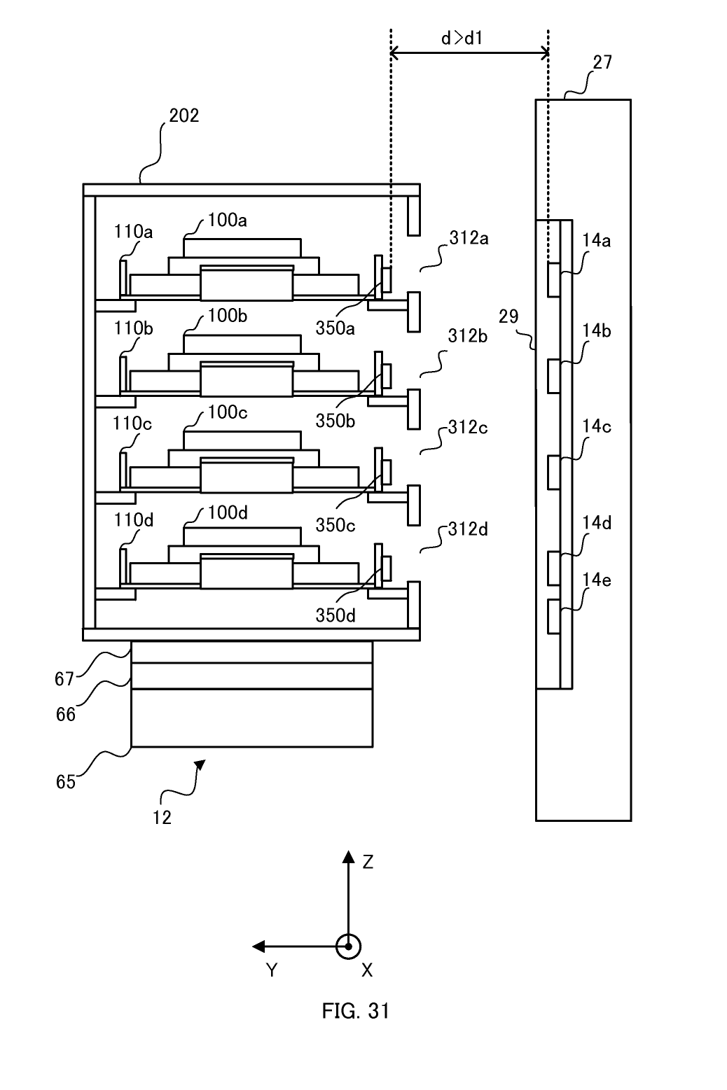

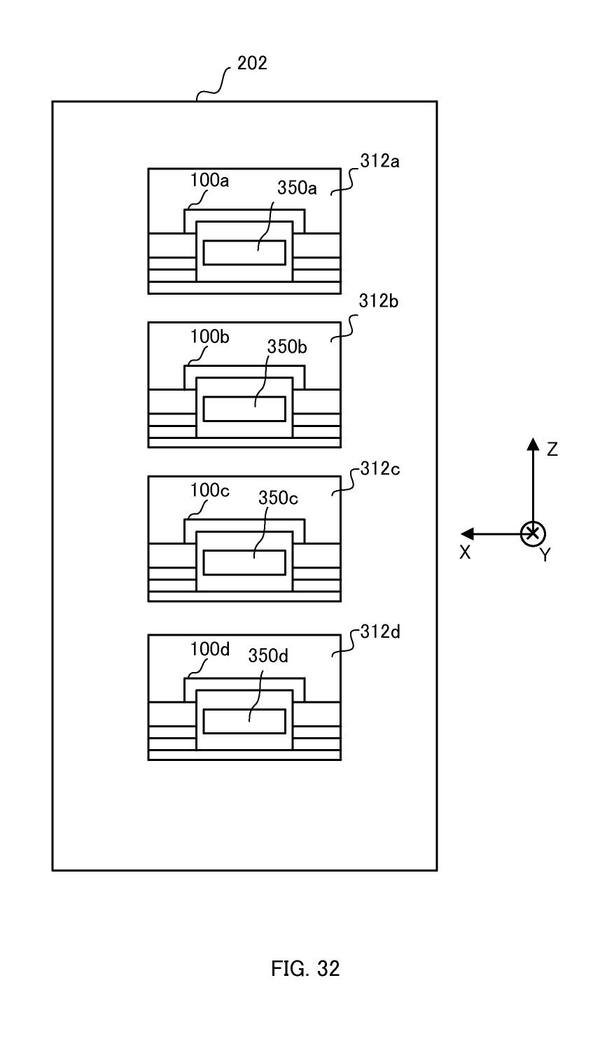

It may be necessary to increase the distance d between the antenna 14 and the wireless tag 350 considering the structures of the stacker 201 and the incubator 11. In the case where the distance d is greater than the predetermined distance (for example, dl), even though the antenna 14 having high radio wave directivity is used, crosstalk might be caused by diffraction of radio waves. In this case, the stacker 202 having a plurality of opening portions 312a to 312d (small windows) as depicted in FIGS. 31 and 32 may be used in place of the stacker 201. With the use of such a stacker 202, the influence of the diffraction of radio waves can be suppressed, thereby being able to prevent crosstalk.

The incubator 11 and the carrier system 10 according to an embodiment of the present invention have been described hereinabove.

At least two antennas are disposed at different heights in the interior of the carrying in/out door 27 of the incubator 11. Further, in the incubator 11, the antenna 14 and the wireless tag 350 are provided so as to be opposed to each other, and from the result of transmitting/receiving radio waves according to the positional relationship therebetween, the placed object is determined among the stacker (storage member), the tray 110 (holding member), and the container 100. Then, based on the determination result and the specified storage destination for the placed object, if it is not appropriate to carry the placed object to the specified storage destination, a warning is given. Thus, the incubator 11 can safely carry the container 100 or the stacker 200.

In an embodiment of the present invention, whether the placed object placed on the carrier table 12 is the container 100 or the stacker 200 is determined, and thereafter the placed object is carried. Accordingly, the carrier system 10 can safely carry the container 100 or the stacker 200.

Further, the containers 100a to 100d stored in the stacker 200 are attached with the wireless tags 350a to 350d storing the identification information capable of uniquely identifying the containers 100a to 100d. Thus, when the reader/writer 13 reads the information of these wireless tags 350a to 350d, it can be determined that the carrying target is the stacker 200.

Further, the wireless tag 350 has recorded therein the storage data indicative of the storage destination. Thus, even after the container 100 is once taken out to the exterior of the culture chamber 22, the container 100 can be returned to the original place with reliability.

Further, as depicted in FIG. 13, since the antenna 14e is disposed lower than the opening portion 310d of the stacker 200, the side face of the stacker 200 shields against radio waves from the antenna 14e. Thus, it can be prevented that the antenna 14e erroneously receives the information of the wireless tag 350d.

Further, as depicted in FIG. 21, space saving can be achieved by disposing the antenna 14e so as to cover a part of the antenna 14d.

Further, the carrying target can be determined with reliability by recording the information indicative of “stacker” or “container” in the wireless tag 350.

Further, in an embodiment of the present invention, it is determined whether the carrying target is the predetermined object that is to be stored at the specified storage destination (for example, process 102 in FIG. 20), and thus the carrying target can be carried more reliably.

The above embodiments of the present invention are simply for facilitating the understanding of the present invention and are not in any way to be construed as limiting the present invention. The present invention may variously be changed or altered without departing from its spirit and encompass equivalents thereof.

For example, the wireless tag 350 may be directly attached to the container 100. Further, a tag such as a bar code may be used in place of the wireless tag.

Furthermore, the carrier device control unit 502 may adjust, for example, the speed or movable range of the carrier device 16 based on the result of the determination of the carrying target. Specifically, when the carrying target is the stacker, the carrier device control unit 502 may move the carrier device 16 at a predetermined speed V1; and when the carrying target is the container, the carrier device control unit 502 may move the carrier device 16 at the predetermined speed V2 (>V1).

What is claimed is:

1. An incubator configured to automatically carry a container to a specified storage destination and culture a cell or a microbe in the container at the storage destination, the incubator comprising:
a mount table configured to have placed thereon the container with a tag; a holding member with the tag at the same height as a height of the tag of the container, the holding member configured to hold the container; or a storage member configured to store the holding members in a multi-stage manner in a vertical direction so that the tag is positioned at a height different from the height of the tag of the container or the holding member;
a carrying-in door for carrying the container, the holding member, or the storage member into the incubator, the carrying-in door provided on one side face of the incubator, the carrying-in door including in an interior thereof a first antenna disposed at the same height as the height of the tag of the container or the holding member; and a second antenna disposed at the same height as the height of the tag of the holding member stored in the storage member;
a discrimination unit configured to, in a state where the container, the holding member, or the storage member is placed on the mount table and the carrying-in door is open,
determine that the container or the holding member is placed on the mount table if radio waves can be transmitted/received between the first antenna and the tag of the container or the holding member, with the first antenna and the tag of the container or the holding member being arranged so as to be opposed to each other, and
determine that the storage member is placed on the mount table if radio waves can be transmitted/received between the second antenna and the tag of the holding member stored in the storage member, with the second antenna and the tag of the holding member stored in the storage member being arranged so as to be opposed to each other; and
a warning unit configured to give warning
if the discrimination unit determines that the container or the holding member is placed on the mount table and the specified storage destination is a place for storing the storage member, or
if the discrimination unit determines that the storage member is placed on the mount table and the specified storage destination is a place for storing the holding member.

2. The incubator according to claim 1, wherein
the storage member is made of a radio wave shielding material, and has an opening portion on one side face thereof to allow radio waves to pass therethrough,
the first antenna is opposed to the tag of the container or the holding member via the opening portion, and
the second antenna is opposed to the tag of the holding member stored in the storage member via the opening portion.

3. A carrier system comprising:
a mount table configured to have placed thereon a container or a storage member configured to store a plurality of containers;
a discrimination unit configured to determine whether an object placed on the mount table is the container or the storage member; and
a carrying unit configured to,
when it is determined that the placed object is the storage member, carry the storage member placed on the mount table to a storage destination for the storage member to be stored in storage via a carrying-in port provided in the storage, and
when it is determined that the placed object is the container, carry the container placed on the mount table to the storage destination for the storage member to be stored in the storage via the carry-in port.

4. The carrier system according to claim 3, wherein
the container is placed on the mount table in a state of being held by the holding member, and is stored in the storage member in a state of being held by the holding member,
the container or the holding member is attached with a tag having recorded therein identification information capable of uniquely identifying the container,
the storage member is configured to store, in a multi-stage manner, the plurality of holding members configured to hold the plurality of containers, respectively, and wherein
the discrimination unit includes:
one reading device provided at a position corresponding to a position of the tag attached to the container or the holding member at a time when the container and the holding member are placed on the mount table, the one reading device configured to read the identification information recorded in the tag of the container or the holding member placed on the mount table;
a plurality of reading devices provided at positions corresponding to positions of the tags respectively attached to the containers or the holding members stored in the storage member at a time when the storage member is placed on the mount table, the plurality of reading devices configured to read the identification information respectively recorded in the tags; and
a discrimination device configured to, when the one reading device reads the identification information recorded in the tag, determine that the placed object is the container, and when the plurality of reading devices read the identification information respectively recorded in the tags, determine that the placed objet is the storage member.

5. The carrier system according to claim 4, wherein
the tag is a writable wireless tag, and
the carrier system further includes:
one writing device configured to, if the container is placed on the mount table, write a storage destination of the container placed on the mount table, into the tag of the container or the holding member placed on the mount table; and
a plurality of writing devices configured to, if the storage member is placed on the mount table, write respective storage destinations of the plurality of containers stored in the storage member, into the tags respectively attached to the containers or the holding members stored in the storage member.

6. The carrier system according to claim 3, wherein
the container is placed on the mount table in a state of being held by the holding member, and is stored in the storage member in a state of being held by the holding member,
the container or the holding member is attached with a wireless tag having recorded therein identification information capable of uniquely identifying the container,
the storage member is made of a radio wave shielding material, and is a member in a box shape configured to store the holding member configured to hold the container, the storage member having a first opening portion on a front face, and having a second opening portion on one side face so as to allow radio waves to pass therethrough to the wireless tag,
the discrimination unit includes:
a first antenna configured to transmit/receive radio waves via the second opening portion to/from the wireless tag attached to the container or the holding member stored in the storage member at a time when the storage member is placed on the mount table;
a second antenna configured to transmit/receive radio waves to/from the wireless tag attached to the container or the holding member at a time when the container and the holding member are placed on the mount table;
a first reading device configured to read, using the first antenna, the identification information recorded in the wireless tag attached to the container or the holding member that is stored in the storage member at a time when the storage member is placed on the mount table;
a second reading device configured to read, using the second antenna, the identification information recorded in the wireless tag of the container or the holding member placed on the mount table; and
a discrimination device configured to determine the placed object based on reading a result of the first and second reading devices,
the second antenna is provided at a position at which the storage member shields against radio waves transmitted from the second antenna when the storage member is placed on the mount table, and wherein
the discrimination device is further configured to determine the placed object is the container when the second reading device reads the identification information recorded in the wireless tag, and determine the placed object is the storage member when the first reading device reads the identification information recorded in the wireless tag.

7. The carrier system according to claim 6, wherein
the second antenna is provided, covering a part of the first antenna, at a position at which the storage member shields against radio waves transmitted from the second antenna when the storage member is placed on the mount table.

8. The carrier system according to claim 3, wherein
the container is placed on the mount table in a state of being held by the holding member, and is stored in the storage member in a state of being held by the holding member,
the container or the holding member is attached with a first tag having recorded therein information indicative of the container,
the storage member is attached with a second tag having recorded therein information indicative of the storage member, and
the discrimination unit includes:
a reading device configured to read information recorded in the tag attached to the placed object placed on the mount table when the container and the holding member, or the storage member is placed on the mount table; and
a discrimination device configured to determine that the placed object is the container if the reading device reads the information indicative of the container, and determine that the placed object is the storage member if the reading device reads the information indicative of the storage member.

9. The carrier system according to claim 3, further comprising:
a determination unit configured to determine whether or not the placed object is the predetermined storage object to be stored at the specified storage destination, based on a determination result of the discrimination unit, first information indicative of a specified storage destination, and second information indicative of a predetermined storage object to be stored at the specified storage destination of the container and the storage member, wherein
the carrying unit configured to,
when it is determined that the placed object is the predetermined storage object to be stored at the specified storage destination and the placed object is the storage member, carry the storage member to the specified storage destination, and
when it is determined that the placed object is the predetermined storage object to be stored at the specified storage destination and the placed object is the container, carry the container to the specified storage destination.

10. The carrier system according to claim 3, further comprising
an input unit configured to specify the storage destination of the placed object placed on the mount table, based on an inputted instruction.
